(19) United States
(12) Patent Application Publication
(10) Pub. No.: US 2020/0069157 A1
(43) Pub. Date: Mar. 5, 2020

(54) HOOD FOR ENDOSCOPE, AND ENDOSCOPE SYSTEM

(71) Applicant: OLYMPUS CORPORATION, Tokyo (JP)

(72) Inventor: Naoto NAKAMURA, Tokyo (JP)

(73) Assignee: OLYMPUS CORPORATION, Tokyo (JP)

(21) Appl. No.: 16/675,356

(22) Filed: Nov. 6, 2019

Related U.S. Application Data
(63) Continuation of application No. PCT/JP2018/016370, filed on Apr. 20, 2018.

(30) Foreign Application Priority Data
May 10, 2017 (JP) 2017-094238

(51) Int. Cl. A61B 1/00; A61B 1/015

(52) U.S. Cl. CPC A61B 1/00137; A61B 1/015

(57) ABSTRACT

A hood for endoscope is attached to an endoscope that includes an insertion portion, a distal end portion, and a conduit, the hood for endoscope including a frame mounted to an outer circumference of a distal end portion, a discharge opening that communicatively connects an inner circumferential surface and an outer circumferential surface of the frame and is provided to be slanted to be oriented in a longitudinal axis direction, and a straightening plate with which a gas ejected from the discharge opening towards a front side in the longitudinal axis direction collides and which guides the gas from a flowing orientation of the gas in the longitudinal axis direction to the flowing orientation of the gas in a radial direction in such a manner that the gas is caused to pass through the discharge opening from an inside of the frame and flow towards an outside of the frame.

### CROSS REFERENCE TO RELATED APPLICATION

This application is a continuation application of PCT/JP2018/016370 filed on Apr. 20, 2018 and claims benefit of Japanese Application No. 2017-094238 filed in Japan on May 10, 2017, the entire contents of which are incorporated herein by this reference.

### BACKGROUND OF INVENTION

### 1. Field of the Invention

The present invention relates to a hood for endoscope which is attached to an endoscope, and an endoscope system.

### 2. Description of the Related Art

In an endoscope system including an endoscope used in a medical field, the following procedure of a cautery apparatus is well known. An insertion portion of the endoscope is inserted into a living body serving as an object. In a state where a treatment target tissue in a living tissue in the living body is observed under an endoscope observation, the cautery apparatus allowed to be inserted into a treatment instrument insertion conduit via a treatment instrument insertion opening provided in an operation portion of the endoscope is caused to protrude from an opening provided in the insertion portion, and energy is supplied from the protruding cautery apparatus to the treatment target tissue to detach and cut off the treatment target tissue from the living tissue.

In one example, an ESD (endoscopic submucosal dissection) procedure, for example, is well-known in an endoscopic treatment for removing a lesion site such as a cancer tissue in the living body under the endoscope observation.

More specifically, the ESD procedure is known as a treatment as follows. For example, the insertion portion of the endoscope is inserted into the living body. In a state where the cancer tissue existing in the living body is set in an observation field of view of the endoscope, the cautery apparatus, for example, a high-frequency knife allowed to be inserted into the treatment instrument insertion conduit of the endoscope is caused to protrude to a front side in a longitudinal axis direction from a distal end of the insertion portion. Thereafter, the insertion portion is moved back and forth in the longitudinal axis direction, and the cancer tissue previously lifted by injection of a dedicated-use liquid is removed using the high-frequency knife

Note that the following configuration is well known in a procedure of the cautery treatment such as the ESD procedure. A tubular hood for endoscope is attached to an outer circumference on a distal end side of the insertion portion. The hood for endoscope keeps a distance between an objective lens serving as an observation member and the treatment target tissue constant, and also, when a skin-like tissue in the lesion site in the treatment target tissue which is scraped by the high-frequency knife, for example, is peeled, the hood for endoscope avoids covering of the lesion site with the skin-like tissue to expose the lesion site.

During the procedure such as the ESD procedure for dissecting the living tissue and also coagulating bleeding, mucosa and fat turn into a fluid containing solid particle components, more specifically, a mist state as a gas containing components derived from the living tissue along with dissection and coagulation performed when a high-frequency current is applied from the high-frequency knife, for example, to the cancer tissue.

As a result, particularly in a procedure over a prolonged period of time such as the ESD procedure, since a small space tends to be filled with mist, and the mist is also more likely to adhere to the objective lens, a situation may occur where the observation field of view of the endoscope is interrupted and becomes blurred.

For this reason, to secure the observation field of view in the ESD procedure, it is desirable to use a technique for sucking the gas containing the components derived from the living tissue from an air suction opening by using an air suction conduit. Note that a configuration in which the air suction conduit also functions as a conduit for treatment instrument insertion is also well known.

Japanese Patent Application Laid-Open Publication No. 2013-165791 discloses a configuration of an endoscope apparatus that feeds a gas from an opening section of an air feeding conduit formed in a surrounding of an objective lens on a distal end surface in an insertion portion of an endoscope to a front side in the longitudinal axis direction of the insertion portion towards the treatment target tissue to avoid adhesion of a suspended matter in the living body to an objective lens and also avoid deterioration of the observation field of view.

### SUMMARY OF THE INVENTION

A hood for endoscope according to one embodiment of the present invention is attached to an endoscope that includes an insertion portion inserted in a longitudinal axis direction from a distal end side into an object, a distal end constituent member that is provided on the distal end side of the insertion portion and has an opening section, and a conduit that is provided in the insertion portion and also communicatively connects an inside and an outside of the object via the opening section, the hood for endoscope including a frame mounted to an outer circumference of the distal end constituent member, a cutout opening that is provided in a position of the frame where the cutout opening protrudes to a front side in the longitudinal axis direction from the distal end constituent member when the frame is attached to the distal end constituent member, communicatively connects an inner circumferential surface and an outer circumferential surface of the frame, and provided to be slanted to be oriented in the longitudinal axis direction, and a straightening member with which a fluid ejected from the opening section towards the front side in the longitudinal axis direction collides and which guides the fluid from a flowing orientation of the fluid in the longitudinal axis direction to the flowing orientation of the fluid in a direction intersecting with the longitudinal axis direction in such a manner that the fluid is caused to pass through the cutout opening from an inside of the frame and flow towards an outside of the frame.

A hood for endoscope according to another embodiment of the present invention is attached to an endoscope that includes an insertion portion inserted in a longitudinal axis direction from a distal end side into an object, a distal end constituent member that is provided on the distal end side of the insertion portion and has an opening section, and a conduit that is provided in the insertion portion and also communicatively connects an inside and an outside of the object via the opening section, the hood for endoscope including a frame mounted to an outer circumference of the distal end constituent member, a cutout opening that is provided in a position of the frame where the cutout opening protrudes to a front side in the longitudinal axis direction from the distal end constituent member when the frame is attached to the distal end constituent member, and communicatively connects an inner circumferential surface and an outer circumferential surface of the frame, and a straightening member with which a fluid ejected from the opening section towards the front side in the longitudinal axis direction collides and which guides the fluid from a flowing orientation of the fluid in the longitudinal axis direction to the flowing orientation of the fluid in a direction intersecting with the longitudinal axis direction in such a manner that the fluid is caused to pass through the cutout opening from an inside of the frame and flow towards an outside of the frame, the straightening member being arranged with a space between the straightening member and the cutout opening.

An endoscope system according to one embodiment of the present invention includes an endoscope including an insertion portion inserted in a longitudinal axis direction from a distal end side into an object, a distal end constituent member that is provided on the distal end side of the insertion portion and has an opening section, and a conduit that is provided in the insertion portion and also communicatively connects an inside and an outside of the object via the opening section, and a hood for endoscope which is attached to the endoscope and includes a frame mounted to an outer circumference of the distal end constituent member, a cutout opening that is provided in a position of the frame where the cutout opening protrudes to a front side in the longitudinal axis direction from the distal end constituent member when the frame is attached to the distal end constituent member, communicatively connects an inner circumferential surface and an outer circumferential surface of the frame, and provided to be slanted to be oriented in the longitudinal axis direction, and a straightening member with which a fluid ejected from the opening section towards the front side in the longitudinal axis direction collides and which guides the fluid from a flowing orientation of the fluid in the longitudinal axis direction to the flowing orientation of the fluid in a direction intersecting with the longitudinal axis direction in such a manner that the fluid is caused to pass through the cutout opening from an inside of the frame and flow towards an outside of the frame.

### BRIEF DESCRIPTION OF THE DRAWINGS

FIG. 1 schematically illustrates a configuration of an endoscope system according to a first embodiment;

FIG. 2 is a partially enlarged perspective view of a distal end portion of an insertion portion of an endoscope in FIG. 1 to which a hood for endoscope is attached;

FIG. 3 is a perspective view of the hood for endoscope in FIG. 2 as viewed from an arrow III in FIG. 2;

FIG. 4 is a front view of a distal end surface in the distal end portion of the insertion portion of the endoscope and the hood for endoscope in FIG. 2 as viewed from an arrow IV in FIG. 2;

FIG. 5 is a partial cross sectional view of the distal end portion and the hood for endoscope along a line V-V in FIG. 4;

FIG. 6A is a partial cross sectional view illustrating a state where a distal end portion to which the hood for endoscope in FIG. 5 is attached is inserted into an object, and feeding of a gas is performed from an air feeding opening;

FIG. 6B is a partial perspective view illustrating a state where the distal end portion to which the hood for endoscope in FIG. 5 is attached is inserted into the object, and the feeding of the gas is performed from the air feeding opening;

FIG. 7 is a partial cross sectional view of the distal end portion to which the hood for endoscope is attached, illustrating a state where a submucosal layer in a lesion site is dissected using a high-frequency knife;

FIG. 8 is a front view of the distal end surface in the distal end portion of the insertion portion, illustrating a modification of a shape of a first straightening member in FIG. 2;

FIG. 9 is a front view of the distal end surface in the distal end portion of the insertion portion, illustrating a modification different from FIG. 8 of the shape of the first straightening member in FIG. 2;

FIG. 10 is a front view illustrating a modification in which a concave section is formed on an outer circumference of the distal end portion of the insertion portion of the endoscope in FIG. 2, and a convex section that is fitted into the concave section is formed on an inner circumferential surface of the hood for endoscope;

FIG. 11 is a perspective view illustrating a modification in which an indicator is provided on an outer circumferential surface of the hood in FIG. 2;

FIG. 12 is a perspective view illustrating a modification in which a notch is provided on the outer circumferential surface of the hood in FIG. 2;

FIG. 13 is a front view illustrating a modification in which a protrusion for positioning the hood for endoscope is provided on the distal end surface in the distal end portion of the insertion portion of the endoscope in FIG. 2;

FIG. 14 is a partial cross sectional view of the hood for endoscope along a line XIV-XIV in FIG. 5;

FIG. 15 is a partial cross sectional view of the hood for endoscope illustrating a modification of an air feeding hole formed in a second straightening member in FIG. 14;

FIG. 16 is a partial cross sectional view of the hood for endoscope illustrating a modification different from FIG. 15 of the air feeding hole formed in the second straightening member in FIG. 14;

FIG. 17 is a partial cross sectional view schematically illustrating a state where a cutout opening of the frame of the hood for endoscope in FIG. 2 is covered with the lesion site;

FIG. 18 is a partial cross sectional view schematically illustrating a modification of a shape of the cutout opening different from FIG. 17 together with the lesion site;

FIG. 19 is a partial cross sectional view schematically illustrating a modification in which the convex section is provided in a vicinity of the cutout opening in FIG. 18 together with the lesion site;

FIG. 20 is a partial cross sectional view of the hood for endoscope illustrating a modification in which the air feeding hole in FIG. 2 and the cutout opening are formed in the frame in an integrated manner; and

FIG. 21 is a perspective view of a configuration in which the first straightening member and the second straightening member in the hood for endoscope in FIG. 3 are constituted as a single straightening member.

### DETAILED DESCRIPTION OF THE PREFERRED EMBODIMENTS

Hereinafter, embodiments of the present invention are described with reference to the drawing. Note that the drawings are schematic drawings, and a relationship between a thickness and a width of each of members, and a thickness ratio and the like of each of the members are different from actual configurations. A part where a mutual dimensional relationship or ratio is different between mutual drawings is included of course.

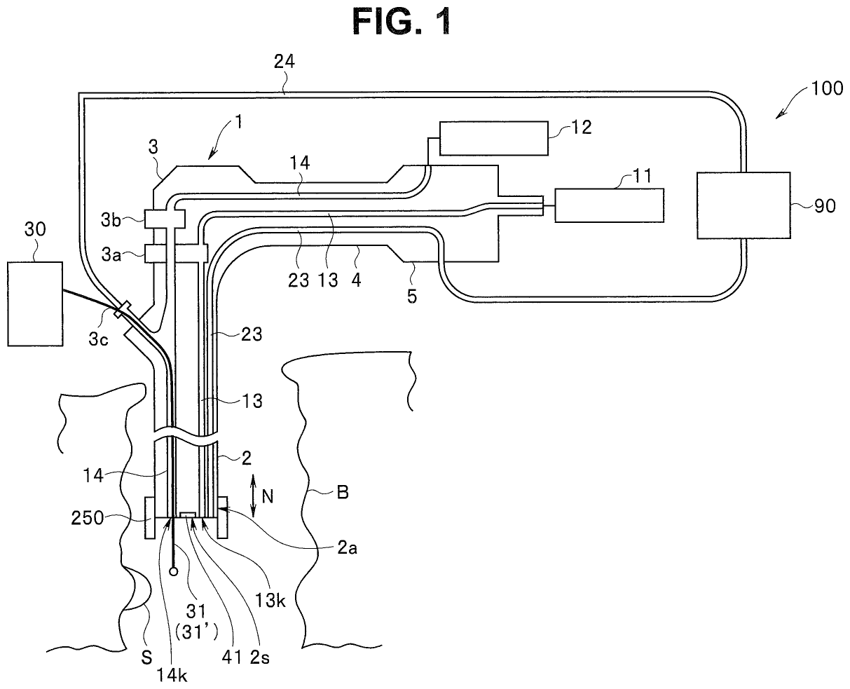

FIG. 1 schematically illustrates a configuration of an endoscope system according to the present embodiment.

As illustrated in FIG. 1, a main portion of an endoscope system 100 is configured of an endoscope 1 and a hood for endoscope 250 attached to the endoscope 1.

A main portion of the endoscope 1 is configured by including an insertion portion 2 inserted in a longitudinal axis direction N from a distal end side into an object B (a lumen of a living body, as an example, a digestive tract), an operation portion 3 communicatively disposed at a proximal end of the insertion portion 2, a universal cord 4 extended from the operation portion 3, and a connector 5 provided at an extension end of the universal cord 4 and detachably attached to an external apparatus.

An air feeding conduit 13 is provided in the endoscope 1, the air feeding conduit 13 communicatively connecting an inside and an outside of the object B and including a distal end opened in a distal end surface 2s of a distal end portion 2a serving as a distal end constituent member provided on the distal end side of the insertion portion 2 as an air feeding opening 13k serving as an opening section, and a proximal end opened in the connector 5. Note that the air feeding conduit 13 is connected to an air feeding pump 11 in the connector 5.

The air feeding pump 11 is configured to supply a predetermined amount of gas into the object B when an operator performs a switching operation on an air feeding switch 3a that is provided in the operation portion 3 and switches the presence or absence of air feeding to the air feeding conduit 13.

An air suction conduit 14 is provided in the endoscope 1, the air suction conduit 14 including a distal end opened in the distal end surface 2s as an air suction opening 14k, and a proximal end opened in the connector 5, and part of the air suction conduit 14 being opened in a forceps opening 3c in the operation portion 3.

Note that the air suction conduit 14 is connected to an air suction pump 12 in the connector 5. The air suction opening 14k may be opened not only in the distal end surface 2s but also in a mid-stream position in the insertion portion 2.

A high-frequency treatment instrument 31 or 31′ of a cautery apparatus 30 inserted into the object B is detachably mounted to the air suction conduit 14 via the forceps opening 3c together with the insertion portion 2.

A distal end side of the high-frequency treatment instrument 31 or 31′ protrudes into the object B via the air suction opening 14k and is used for treatment with respect to a lesion site S in a treatment target tissue in the object B.

The air suction pump 12 is provided in the air suction conduit 14 in the operation portion 3 and configured to feed a predetermined amount of gas from the object B to the outside of the object when the operator performs a switching operation on an air suction switch 3b that switches the presence or absence of air suction by the air suction conduit 14.

An air feeding conduit 23 is provided in the endoscope 1, the air feeding conduit 23 communicatively connecting the inside and the outside of the object B and including a distal end opened in the distal end surface 2s of the distal end portion 2a, and a proximal end opened in the connector 5. The air feeding conduit 23 is separately provided, for example, from the air feeding conduit 13.

The air feeding conduit 23 is connected to a recirculation pump 90 in the connector 5.

The recirculation pump 90 is connected to a control portion in the external apparatus (which are neither illustrated in the drawings) and configured to avoid adhesion of a suspended matter in the living body on an objective lens 41 which is described below and also avoid deterioration in an observation field of view of the objective lens 41 by continuously supplying a filtered gas into the object B via the air feeding conduit 23, the filtered gas being obtained by continuously sucking the gas via air suction conduits 14 and 24 to be fed to the outside of the object B by an operation control of the control portion independently from an operation of the operator.

The endoscope system 100 includes the air suction conduit 24 that has the distal end connected to the forceps opening 3c and the proximal end connected to the recirculation pump 90.

In other words, the air suction conduit 24 is branched from the air suction conduit 14 and also used as the air suction conduit 14 in at least the insertion portion 2. Note that of course, the air suction conduit 24 may also be separately provided from the air suction conduit 14.

The recirculation pump 90 sucks a gas including particles that disturbs the field of view of the endoscope 1 which are generated when the high-frequency treatment instrument 31 or 31′ of the cautery apparatus 30 supplies the energy to the living body.

In other words, an air feeding mechanism and an air suction mechanism which filter the gas fed from the object to be supplied into the object again are integrated in the recirculation pump 90.

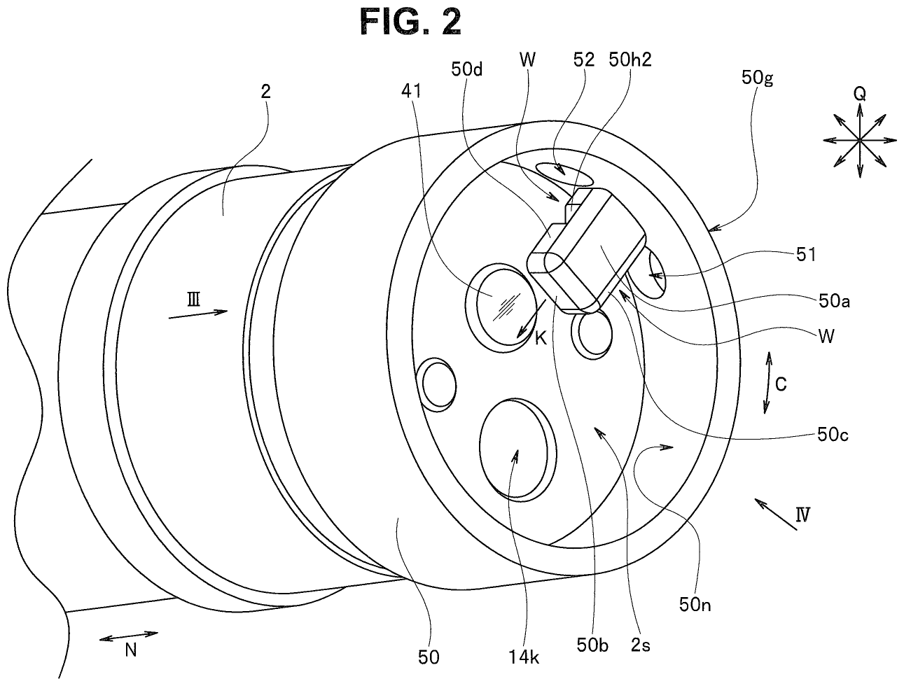

Next, a configuration of the hood for endoscope 250 will be described with reference to FIGS. 2 to 20.

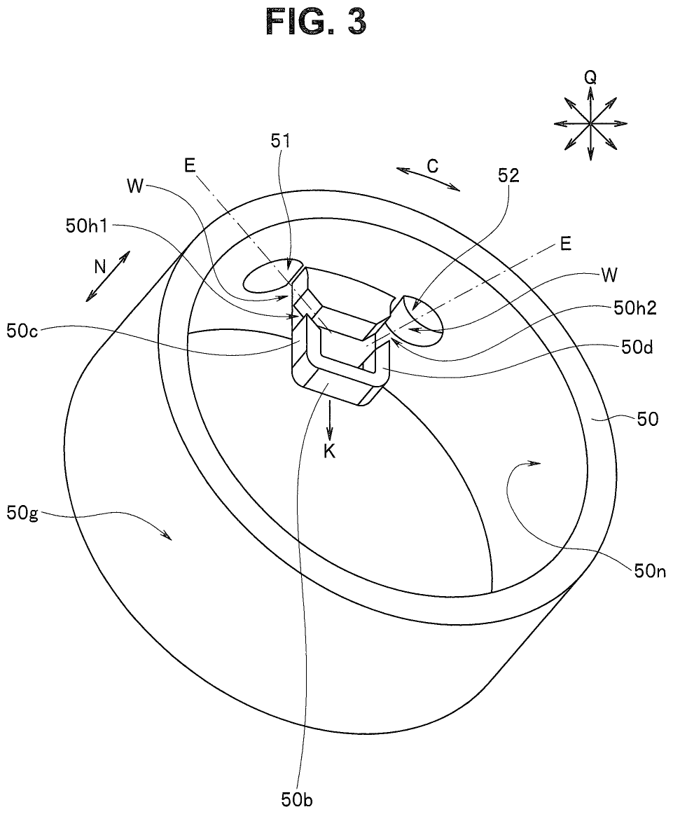

FIG. 2 is a partially expanded perspective view in which the hood for endoscope is attached to the distal end portion of the insertion portion of the endoscope in FIG. 1, and FIG. 3 is a perspective view of the hood for endoscope in FIG. 2 as viewed from an arrow III in FIG. 2.

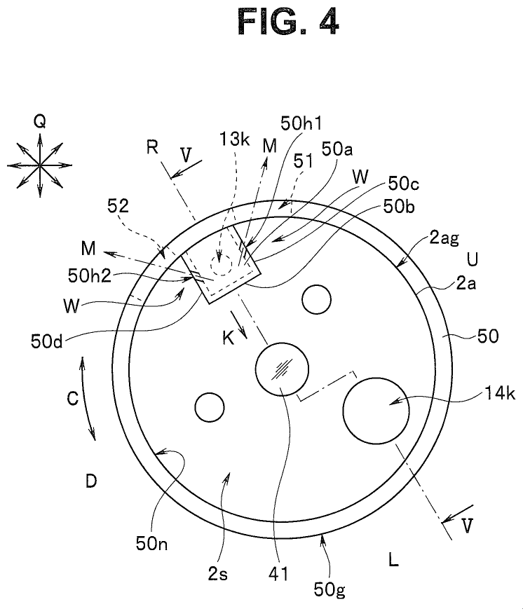

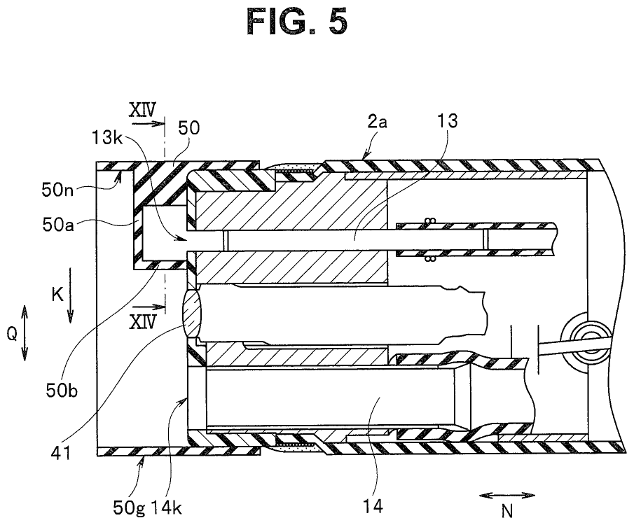

FIG. 4 is a front view of the distal end surface in the distal end portion of the insertion portion of the endoscope in FIG. 2 and the hood for endoscope as viewed from an arrow IV in FIG. 2, and FIG. 5 is a partial cross sectional view of the distal end portion and the hood for endoscope along a line V-V in FIG. 4.

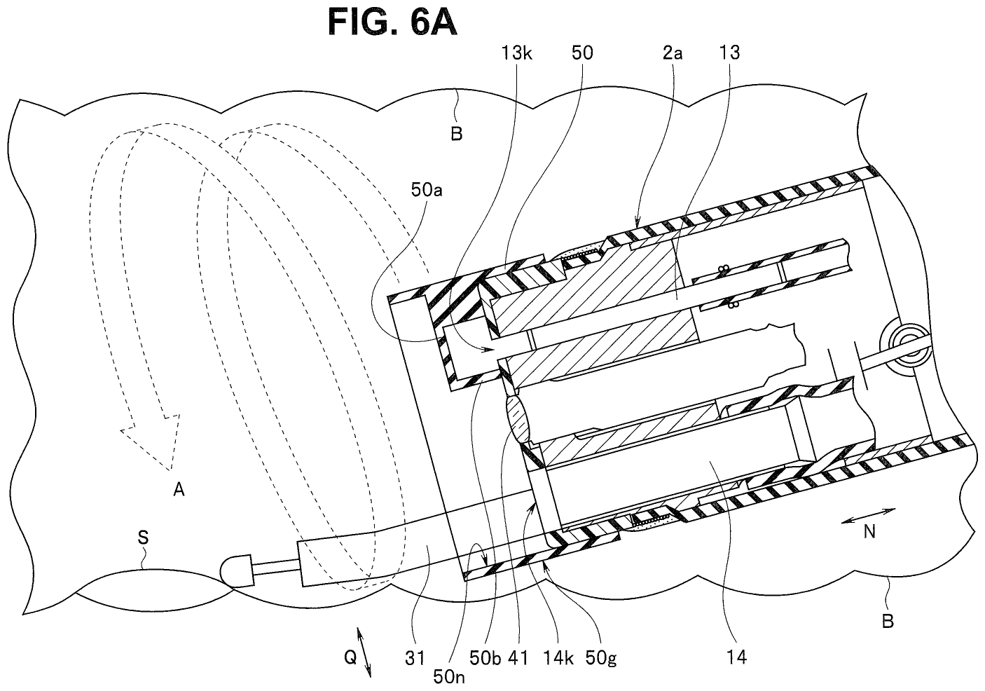

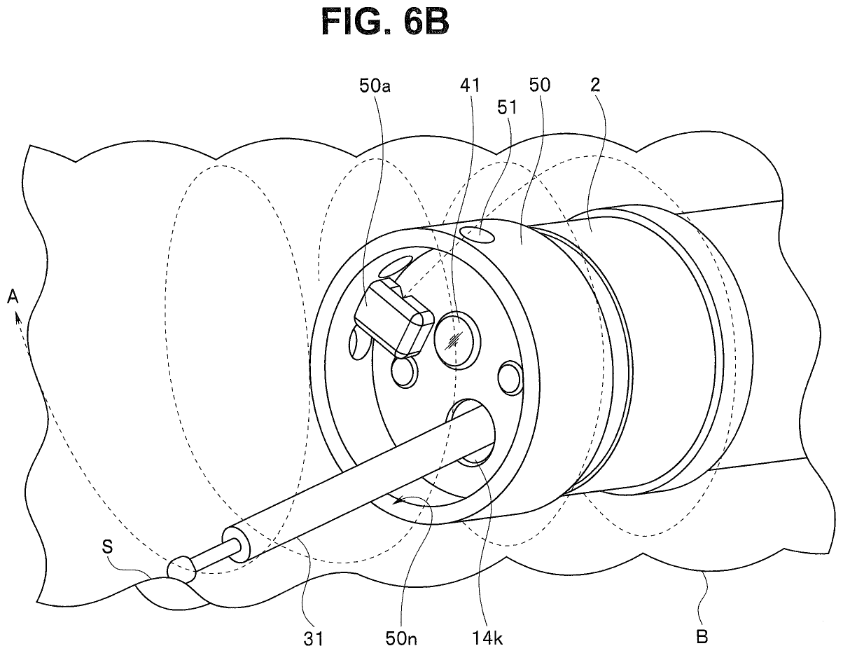

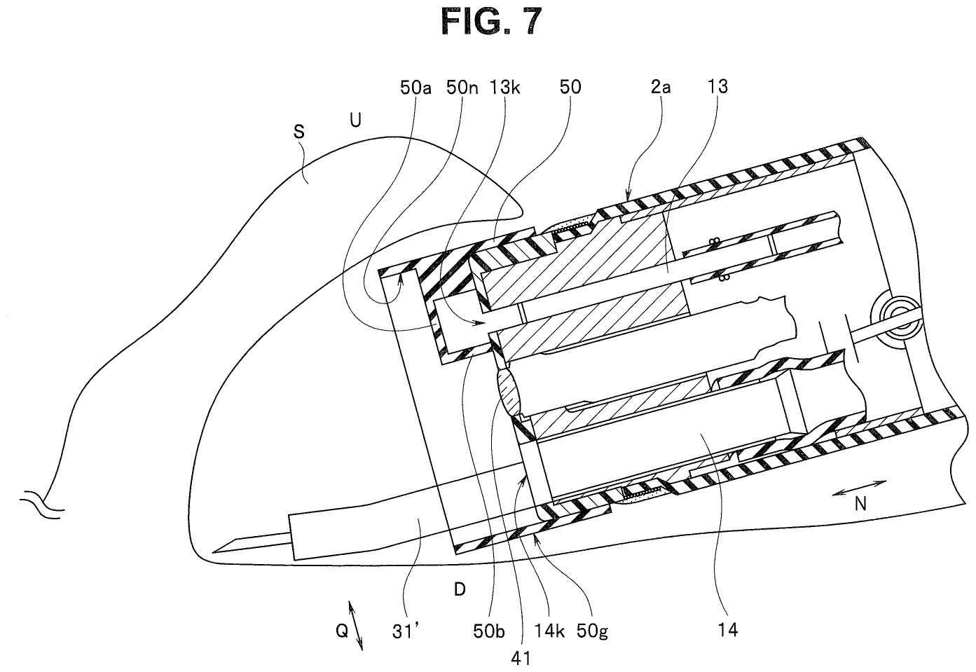

FIG. 6A is a partial cross sectional view illustrating a state where the distal end portion to which the hood for endoscope in FIG. 5 is attached is inserted into the object, and feeding of a gas is performed from the air feeding opening, FIG. 6B is a partial cross sectional view illustrating a state where the distal end portion to which the hood for endoscope in FIG. 5 is attached is inserted into the object, and feeding of the gas is performed from the air feeding opening, and FIG. 7 is a partial cross sectional view of the distal end portion to which the hood for endoscope is attached, illustrating a state where a submucosal layer in a lesion site is dissected using a high-frequency knife.

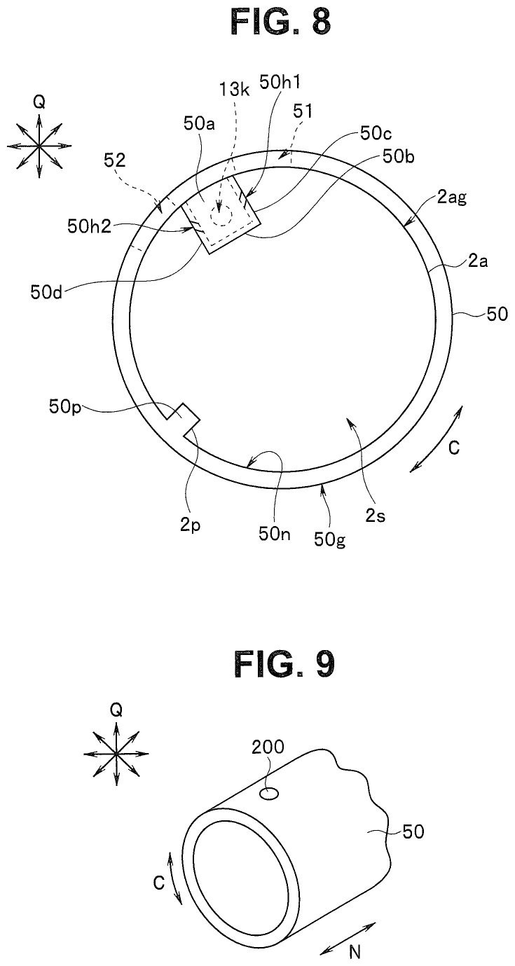

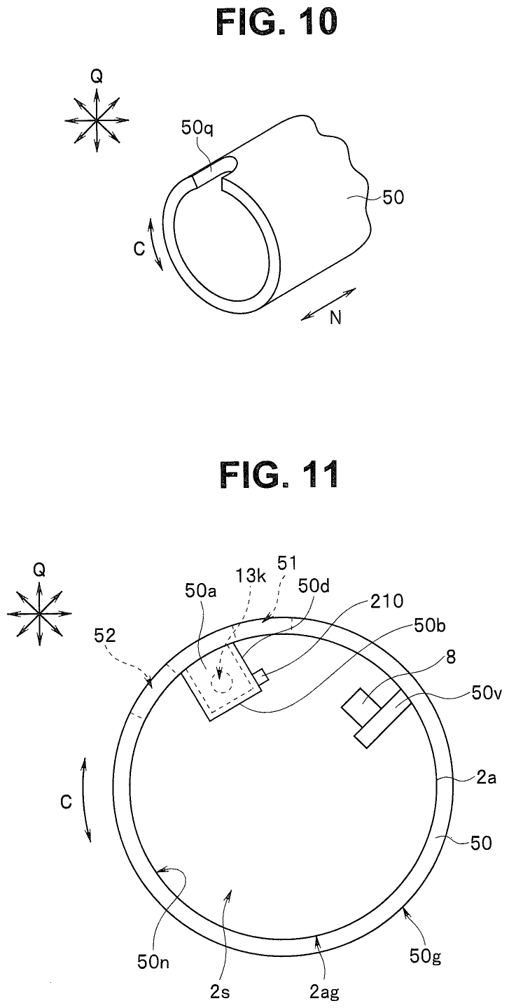

FIG. 8 is a front view illustrating a modification in which a concave section is formed on an outer circumference in the distal end portion of the insertion portion of the endoscope in FIG. 2, and a convex section fitted into the concave section is formed on the inner circumferential surface of the hood for endoscope, FIG. 9 is a perspective view illustrating a modification in which an indicator is provided on the outer circumferential surface of the hood in FIG. 2, and FIG. 10 is a perspective view illustrating a modification in which a notch is provided on the outer circumferential surface of the hood in FIG. 2.

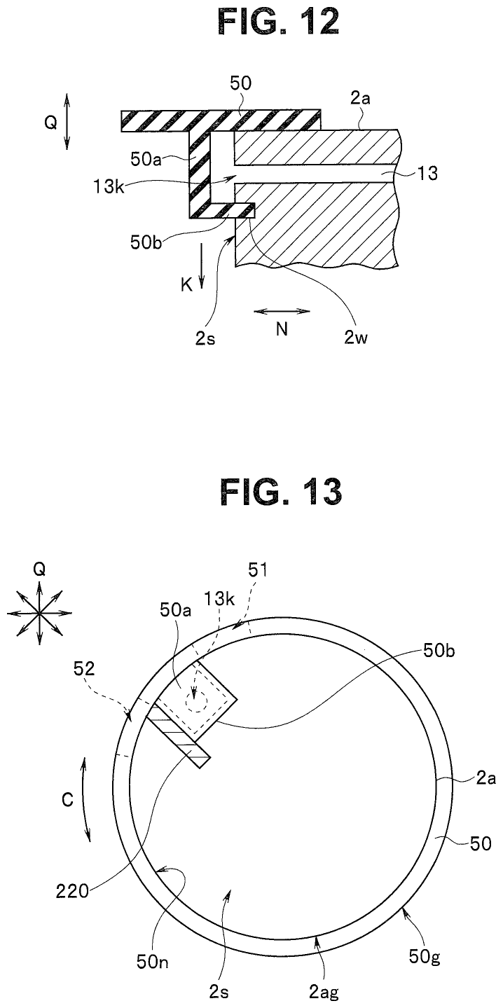

FIG. 11 is a front view illustrating a modification in which a protrusion for positioning the hood for endoscope is provided on the distal end surface in the distal end portion of the insertion portion of the endoscope in FIG. 2, FIG. 12 is a partial cross sectional view illustrating a modification in which the concave section for positioning the hood for endoscope is provided on the distal end surface in the distal end portion of the insertion portion of the endoscope in FIG. 2, and FIG. 13 is a front view illustrating a modification in which the indicator for positioning the hood for endoscope is provided on the distal end surface in the distal end portion of the insertion portion of the endoscope in FIG. 2.

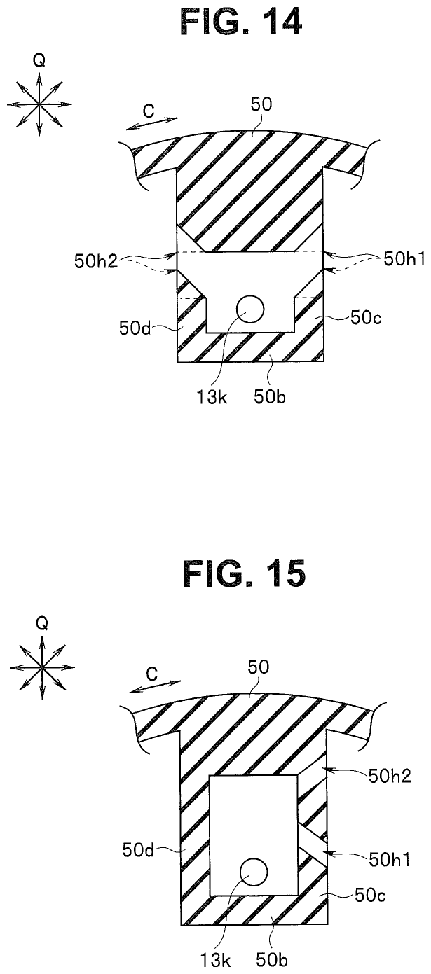

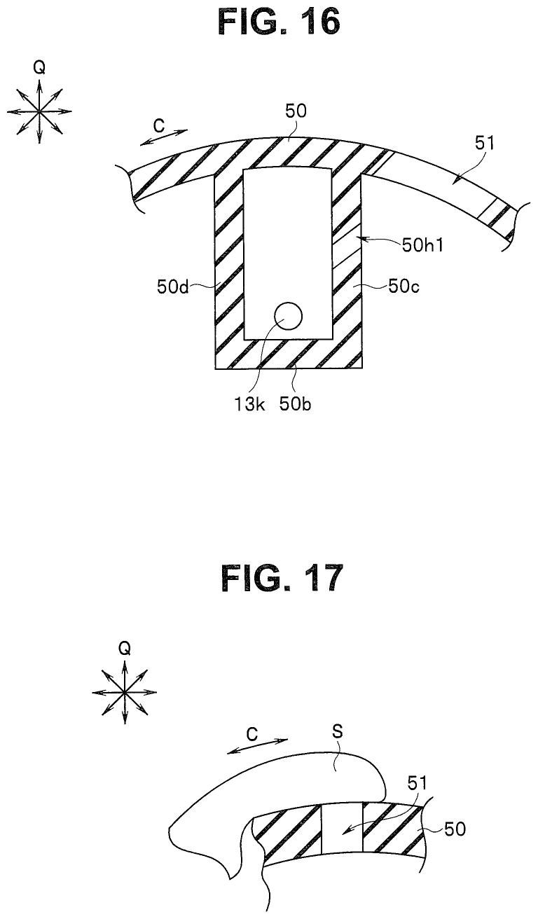

FIG. 14 is a partial cross sectional view of the hood for endoscope along a line XIV-XIV in FIG. 5, FIG. 15 is a partial cross sectional view of the hood for endoscope illustrating a modification of an air feeding hole formed in a second straightening member in FIG. 14, and FIG. 16 is a partial cross sectional view of the hood for endoscope illustrating a modification different from FIG. 15 of the air feeding hole formed in the second straightening member in FIG. 14.

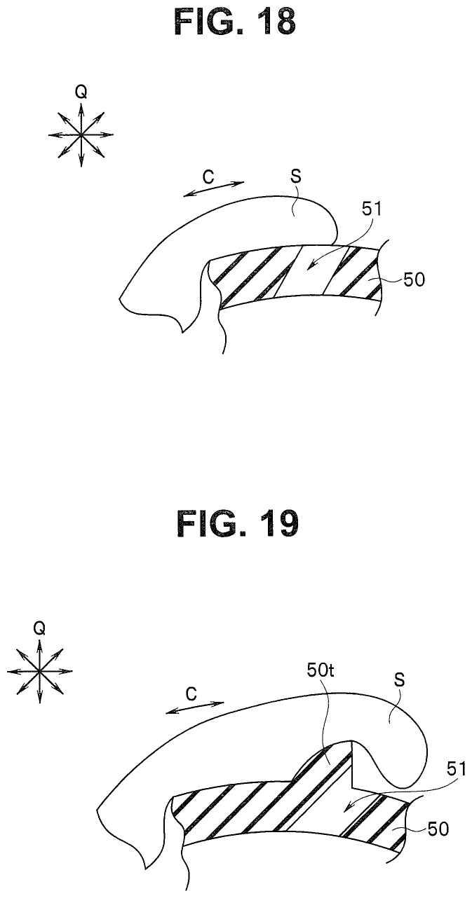

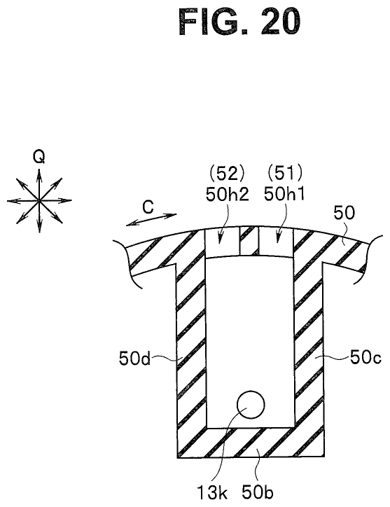

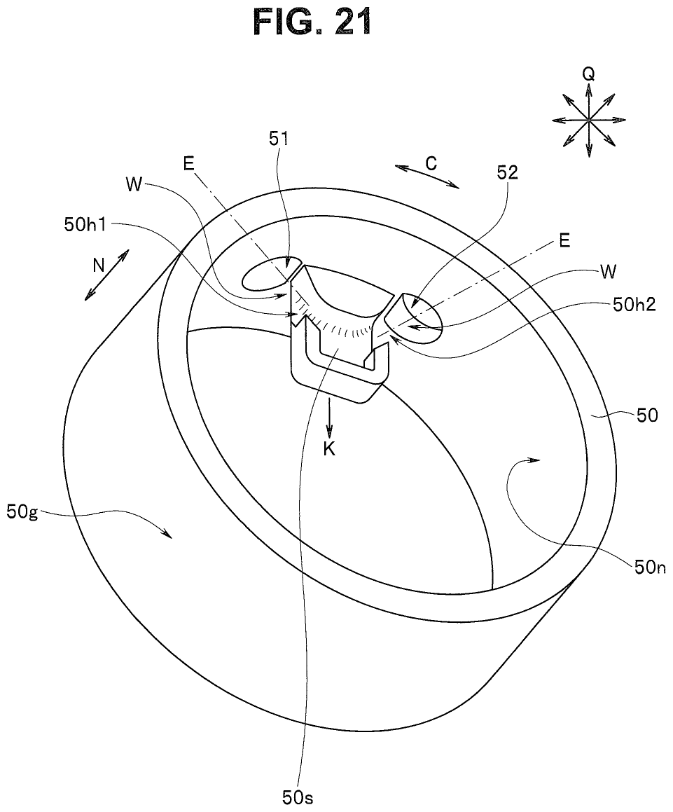

FIG. 17 is a partial cross sectional view schematically illustrating a state where the cutout opening in the frame of the hood for endoscope in FIG. 2 is covered with the lesion site, FIG. 18 is a partial cross sectional view schematically illustrating a modification different from FIG. 17 of the shape of the cutout opening together with the lesion site, FIG. 19 is a partial cross sectional view schematically illustrating a modification in which a convex section is provided in a vicinity of the cutout opening in FIG. 18 together with the lesion site, and FIG. 20 is a partial cross sectional view of the hood for endoscope illustrating a modification in which the air feeding hole in FIG. 2 and the cutout opening are formed in the frame in an integrated manner.

In a procedure of the cautery treatment such as the above-mentioned ESD procedure, after the hood for endoscope (hereinafter, simply referred to as hood) 250 is mounted to the distal end portion 2a, the hood for endoscope 250 is configured to maintain a constant distance between the objective lens 41 provided on the distal end surface 2s which serves as an observation member configured to observe an inside of the object and the treatment target tissue and also, when a skin-like tissue of the lesion site S in the treatment target tissue which is scraped, for example, by the high-frequency treatment instrument 31 is peeled, avoid cover of the lesion site S with the skin-like tissue and expose the lesion site.

As illustrated in FIG. 2, and FIGS. 4 to 7, the hood 250 includes a frame 50 mounted to an outer circumference 2ag of the distal end portion 2a (see FIG. 4).

As illustrated in FIG. 3, the frame 50 is formed to have a tubular shape having a predetermined length along the longitudinal axis in the direction N, and the distal end and the proximal end of the frame 50 are opened.

As illustrated in FIGS. 2 to 4, discharge openings 51 and 52 are formed in the frame 50 in positions protruding to a front side in the longitudinal axis direction N from the distal end portion 2a of the frame 50 when the frame 50 is attached to the distal end portion 2a, the discharge openings 51 and 52 communicatively connecting an inner circumferential surface 50n and an outer circumferential surface 50g of the frame 50 and serving as cutout openings for discharging a gas A in the frame 50 into the object B.

As illustrated in FIGS. 2 to 7, a counter plate 50a serving as a first straightening member that changes a flowing orientation of the gas A from the longitudinal axis direction N to a radial direction Q of the distal end portion 2a corresponding to a direction intersecting with the longitudinal axis direction when the gas A serving as a fluid ejected from the air feeding opening 13k towards the front side in the longitudinal axis direction N collides with the inner circumferential surface 50n of the frame 50 is provided to be located while facing the air feeding opening 13k after the mounting of the frame 50 to the distal end portion 2a. Note that the fluid is not limited to a gas.

In other words, the counter plate 50a is configured to avoid direct feeding of the gas A to the lesion site S located on the front side in the longitudinal axis direction N.

Straightening plates 50b, 50c, and 50d serving as a second straightening member guiding the gas A in a direction M as illustrated in FIG. 4 are provided along the radius in the direction Q continuously to the counter plate 50a while facing the inner circumferential surface 50n such that the gas A the orientation of which is changed by the counter plate 50a passes through the discharge openings 51 and 52 from the inside of the frame 50 and flows from the outer circumferential surface 50g of the frame 50 towards the outside.

The straightening plates 50b, 50c, and 50d guide the gas A the orientation of which is changed by the counter plate 50a in a direction different from a position where an objective lens 41a of the distal end portion 2a is provided.

Note that after the mounting of the frame 50 to the distal end portion 2a, the straightening plate 50b is located between the air feeding opening 13k on the distal end surface 2s and the objective lens 41 in the radial direction Q. In other words, the straightening plate 50b is configured to avoid direct feeding of the gas A to the objective lens 41 in a direction K.

After the mounting of the frame 50 to the distal end portion 2a, as illustrated in FIG. 5, the straightening plates 50b, 50c, and 50d form a closed space between the distal end portion 2a and the hood 250 except for air feeding holes 50h1 and 50h2 that are described below by abutting against the distal end surface 2s in an airtight manner.

As illustrated in FIGS. 2 to 4, the air feeding holes 50h1 and 50h2 that guide the gas A the orientation of which is changed by the counter plate 50a into the frame 50 are respectively formed in the straightening plates 50c and 50d. Note that the air feeding holes 50h1 and 50h2 simultaneously guide the gas A towards the discharge openings 51 and 52.

As illustrated in FIGS. 3 and 14, the air feeding holes 50h1 and 50h2 are respectively formed while facing the discharge openings 51 and 52 to guide the gas A to the discharge openings 51 and 52. For example, in the radial direction Q, the air feeding holes 50h1 and 50h2 are respectively opened along a line in a direction inclined by a predetermined angle from a line in a direction orthogonal to the longitudinal axis direction N.

Note that as represented by dotted lines in FIG. 14, in the radial direction Q, the air feeding holes 50h1 and 50h2 may be respectively opened along a line in direction orthogonal to the longitudinal axis direction.

As illustrated in FIG. 15, depending on formation positions of the discharge openings 51 and 52, the air feeding holes 50h1 and 50h2 may be formed only on the straightening plate 50c.

In other words, the air feeding holes 50h1 and 50h2 may be opened along a line in any direction in the radial direction Q as long as the gas A is not fed in a direction on a side of the objective lens 41.

Note that the counter plate 50a and at least one of the straightening plates 50c and 50d may be formed in the frame 50 in an integrated manner.

The counter plate 50a and the straightening plates 50b, 50c, and 50d may be formed in an integrated manner. When the counter plate 50a, the straightening plates 50b, 50c, and 50d, and the frame 50 are formed in an integrated manner, work process when the hood 250 is formed is facilitated, and also the hood 250 can be thrown away after use.

Of course, the counter plate 50a and the straightening plates 50b, 50c, and 50d may be obtained by respectively fixing separate members to be integrated with one another, or may be obtained by respectively fixing separate members in the frame 50.

The discharge openings 51 and 52 are provided to be adjacent to both arms in a circumferential direction C of the frame 50 in sections where the counter plate 50a, the straightening plates 50b, 50c, and 50d are provided with respect to the frame 50. Note that a reason why the discharge openings 51 and 52 are formed apart from each other in the circumferential direction C is to avoid blocking of the discharge openings 51 and 52 by the same mucosa at the same time.

More specifically, as illustrated in FIG. 3, the discharge openings 51 and 52 are formed to be located on extension lines E of the respective air feeding holes 50h1 and 50h2 with respect to the frame 50.

Note that as illustrated in FIGS. 2 to 4, a space W is formed between the air feeding holes 50h1 and 50h2 and the discharge openings 51 and 52 in the frame 50.

After the mounting of the frame 50 to the distal end portion 2a, as illustrated in FIG. 4, the discharge openings 51 and 52 are positioned so as to be located in one of left and right (LR) in the orientation in the circumferential direction C in which direction references are set in up and down (UD) directions of an image obtained by the objective lens 41.

Note that after the mounting of the frame 50 to the distal end portion 2a, to uniquely regulate an attachment position of the frame 50 in the circumferential direction C with respect to the outer circumference 2ag of the distal end portion 2a, that is, to uniquely regulate the attachment position of the frame 50 that the discharge openings 51 and 52 face in any one of left and right directions in the circumferential direction C, a positioning portion may be provided in at least one of the distal end portion 2a and the frame 50.

More specifically, as illustrated in FIG. 8, the attachment position of the frame 50 in the circumferential direction C may be uniquely regulated when a concave section 2p serving as the positioning portion is formed on the outer circumference 2ag of the distal end portion 2a, a convex section 50p serving as the positioning portion that is fitted into the concave section 2p is formed on the inner circumferential surface 50n of the frame 50, and the convex section 50p is fitted into the concave section 2p when the frame 50 is attached to the distal end portion 2a.

As illustrated in FIG. 9, the attachment position of the frame 50 in the circumferential direction C may also be uniquely regulated when an indicator 200 serving as the positioning portion is provided on a side in the UP direction in the observation field of view of the objective lens 41 in the frame 50, for example, and a person who mounts the frame 50 is notified of the orientation in the circumferential direction C of the frame 50 by way of the indicator 200.

As illustrated in FIG. 10, the attachment position of the frame 50 may also be uniquely regulated when a notch 50q serving as the positioning portion is provided on the side in the UP direction in the observation field of view of the objective lens 41 in the frame 50, for example, and the person who mounts the frame 50 is notified of the orientation in the circumferential direction C of the frame 50 by way of the notch 50q.

Note that the attachment position of the frame 50 in the circumferential direction C may also be uniquely regulated when the notch 50q is fitted into the protrusion or the like formed on the outer circumference 2ag of the distal end portion 2a.

As illustrated in FIG. 11, the attachment position of the frame 50 in the circumferential direction C may also be uniquely regulated when a protrusion 210 serving as the positioning portion is provided on the distal end surface 2s, and the straightening plate 50d serving as the positioning portion abuts against the protrusion 210.

As illustrated in FIG. 11, the attachment position of the frame 50 in the circumferential direction C may also be uniquely regulated when a protrusion 50v serving as the positioning portion which extends in the radial direction Q from the inner circumferential surface 50n of the frame 50 abuts against a nozzle 8 serving as the positioning portion which supplies a fluid to the objective lens 41 provided on the distal end surface 2s.

As illustrated in FIG. 12, the attachment position of the frame 50 in the circumferential direction C may also be uniquely regulated when a concave section 2w serving as the positioning portion is provided on the distal end surface 2s, and part of the straightening plate 50b serving as the positioning portion is fitted into the concave section 2w.

As illustrated in FIG. 13, the attachment position of the frame 50 in the circumferential direction C may also be uniquely regulated when the person who mounts the frame 50 aligns the discharge opening 52 with an indicator 220 serving as the positioning portion formed on the distal end surface 2s.

Note that the embodiment can be applied to not only the configurations illustrated in FIGS. 8 to 13 but also any configuration as long as the attachment position of the frame 50 in the circumferential direction C to the distal end portion 2a can be uniquely regulated.

Positioning for causing the discharge openings 51 and 52 to be located in any one of left and right (LR) after the mounting of the frame 50 to the distal end portion 2a is performed because, as illustrated in FIG. 7, when the treatment target tissue is dissected using a high-frequency knife 31′, and the distal end portion 2a and the hood 250 are brought under the submucosal layer in the lesion site S, in a case where the discharge openings 51 and 52 are formed in any one of up and down directions, the discharge openings 51 and 52 may be blocked by the lesion site S or the submucosal layer.

With regard to the discharge openings 51 and 52, for example, FIGS. 17 to 19 illustrate only the discharge opening 51 as a shape example. In a case where the discharge opening 51 is formed to be like a straight line in the cross section as illustrated in FIG. 17, when the treatment target tissue is dissected using the high-frequency knife 31′, and the distal end portion 2a and the hood 250 are brought under the submucosal layer in the lesion site S as illustrated in FIG. 7, the discharge opening 51 may be blocked by the lesion site S.

Therefore, as illustrated in FIG. 18, when the discharge opening 51 is formed to be inclined to be oriented to a side on the distal end surface 2s in the longitudinal axis direction N, it is preferable that the discharge opening 51 is less likely to be completely blocked by the lesion site S or the submucosal layer.

As illustrated in FIG. 19, when a convex section 50t protruding onto an outer side in the radial direction Q is provided in the frame 50 in a vicinity of the discharge opening 51, since the convex section 50t lifts the lesion site S above the discharge opening 51, the discharge opening 51 is still less likely to be completely blocked by the lesion site S or the submucosal layer.

Note that even if the discharge openings 51 and 52 are completely blocked, as described above, since the space W is formed between the discharge openings 51 and 52 and the air feeding holes 50h1 and 50h2, the gas A is fed in a direction along the inner circumferential surface 50n of the frame 50 and can be supplied into the object B via a distal end opening of the frame 50.

Only one discharge opening may be formed as the discharge opening 51 with respect to the frame 50 as illustrated in FIG. 16, although a probability that the discharge opening is blocked by the lesion site S or the submucosal layer is higher than a probability in a case where a plurality of discharge openings are formed. In this case, it is sufficient when the air feeding hole 50h1 is formed only on the straightening plate 50c.

As illustrated in FIG. 20, a configuration may also be included in which the discharge openings 51 and 52 are also used as the air feeding holes 50h1 and 50h2.

According to the above-mentioned configuration, the gas A ejected to the front side in the longitudinal axis direction from the air feeding opening 13k collides with the counter plate 50a, and the orientation of the gas A is changed in the radial direction Q, and thereafter, the gas A is guided by the straightening plates 50b, 50c, and 50d to flow towards the outside of the frame 50 via the air feeding holes 50h1 and 50h2 and the discharge openings 51 and 52.

Thereafter, as illustrated in FIG. 6, the gas A is fed to the front side in the longitudinal axis direction N while drawing a helical shape in the object B, that is, a convective flow is generated in the object B, and the gas A contacts the above-mentioned mist generated at the time of the cautery treatment on the lesion site S using the high-frequency treatment instrument 31.

Note that at this time, since the gas A is fed from the outside of the outer circumferential surface 50g of the frame 50 in the radial direction Q to the lesion site S while drawing a helical shape, the gas A is more softly fed to the lesion site S as compared with a related art, that is, the gas A is indirectly, neither linearly or directly, fed to the lesion site from the air feeding opening of the distal end surface of the endoscope.

Note that the mist or the like that is moved from a surrounding of the lesion site S by supply of the gas A in the object B is sucked by the air suction conduits 14 and 24 via the air suction opening 14k.

In this manner, according to the present embodiment, it is illustrated that the counter plate 50a and the straightening plates 50b, 50c, and 50d are provided in the frame 50 mounted to the outer circumference 2ag of the distal end portion 2a.

It is also illustrated that the air feeding holes 50h1 and 50h2 that guide the gas A the orientation of which is changed by the counter plate 50a are respectively formed in the straightening plates 50c and 50d.

It is further illustrated that when the frame 50 is attached to the distal end portion 2a, the discharge openings 51 and 52 are located on the extension line E of the air feeding holes 50h1 and 50h2 and also formed in both arms of the counter plate 50a and the straightening plates 50b, 50c, and 50d in the circumferential direction C in the frame 50.

It is also illustrated that the space W is formed between the discharge openings 51 and 52 and the air feeding holes 50h1 and 50h2.

According to this, the counter plate 50a avoids a situation where the gas A ejected from the air feeding opening 13k is directly fed to the lesion site S, the straightening plate 50b also avoids a situation where the gas A is fed to the objective lens 41, and the straightening plates 50c and 50d guide the gas A to the outside of the outer circumferential surface 50g of the frame 50 via the air feeding holes 50h1 and 50h2 and the discharge openings 51 and 52. Thereafter, the gas A is fed to the lesion site S while drawing a helical shape in the object B.

For this reason, deformation or vibration of the lesion site S to which the gas is fed or foaming of a viscous fluid in the surrounding of the lesion site S or the like is not caused by the feeding of the gas unlike a case where the gas A is directly fed into the object B such as the lumen of the living body from the air feeding opening 13k to the front side in the longitudinal axis direction N as in a related art.

In accordance with the configuration according to the present embodiment, since the configuration is included in which, without directly feeding the gas A to the front side of the frame 50, air feeding is performed to the front side after the gas A is taken out to the outside of the outer circumferential surface 50g of the frame 50 via the discharge openings 51 and 52 once, it is possible to more reliably avoid deformation of the lesion site S and foaming of a liquid in the surrounding of the lesion site S.

Therefore, a situation is avoided where the treatment performance on the lesion site S using the high-frequency treatment instrument 31 is decreased or the observation field of view of the objective lens 41 is degraded.

Since the gas A is not directly supplied towards the objective lens 41, contamination filled in the object B is not sprayed on the objective lens 41.

Even if the discharge openings 51 and 52 are blocked by the lesion site S or the like, since the gas A coming into the frame 50 from the air feeding holes 50h1 and 50h2 is fed in the direction along the inner circumferential surface 50n of the frame 50 by the space W, the gas A can be reliably fed from the opening on the front side of the frame 50 to the lesion site S while drawing a helical shape.

With the inclusion of the configuration in which the degradation in the observation field of view or the decrease in the treatment performance can be avoided by using the hood 250 that is mounted as in a related art to the outer circumference 2ag of the distal end portion 2a, increase in a diameter on the distal end side of the insertion portion 2 of the endoscope 1 like a related art is not caused.

Thus, it is possible to provide the hood for endoscope 250 including the configuration with which the increase in the diameter on the distal end side of the insertion portion 2 of the endoscope 1 can be avoided, and also the satisfactory observation field of view of the endoscope 1 at the time of the cautery treatment on the treatment target tissue can be secured, and also provide the endoscope system 100.

According to the above-mentioned embodiment, the configuration including the first straightening member such as the counter plate 50a with which the fluid ejected towards the front side in the longitudinal axis direction N from the opening section such as the air feeding opening 13k collides and which changes this flowing orientation of the fluid from the longitudinal axis direction N to the direction intersecting with the longitudinal axis direction N, and the second straightening member such as the straightening plates 50b, 50c, and 50d guiding the fluid the orientation of which is changed by the first straightening member to pass through the cutout openings (discharge openings 51 and 52) from the inside of the frame 50 and flow towards the outside of the frame 50 has been described, but the configuration of the straightening member configured to change the orientation of the fluid is not limited to a configuration constituted by a plurality of parts as described above.

FIG. 21 is a perspective view of a modification in which the first straightening member and the second straightening member in the hood for endoscope in FIG. 3 are constituted as a single straightening member. For example, as illustrated in FIG. 21, instead of respectively providing the first straightening member and the second straightening member, a configuration may also be adopted which includes a straightening member 50s playing both roles of the first straightening member and the second straightening member at the same time as a single straightening member with which the fluid ejected towards the front side in the longitudinal axis direction N from the opening section such as the air feeding opening 13k collides and which causes the flowing orientation of the fluid to pass through the cutout openings (discharge openings 51 and 52) from the inside of the frame 50 and flow towards the outside of the frame 50.

Even in a configuration in which the straightening member 50s is provided as described above, the gas can be guided in a direction along the inner circumferential surface 50n of the frame 50 such that the situation where the fluid (gas A) ejected from the air feeding opening 13k is directly fed to the lesion site S or the objective lens 41 is avoided, and also the gas A is fed to the lesion site S while drawing a helical shape in the object B such as the lumen of the living body, and the deformation or the vibration of the lesion site S to which the gas is fed or the foaming of the viscous fluid in the surrounding of the lesion site S or the like can be suppressed.

In other words, it is possible to attain similar advantages to those of other embodiments of the present invention in that the situation can be avoided where the treatment performance on the lesion site S using the high-frequency treatment instrument 31 is decreased, or the observation field of view of the objective lens 41 is degraded.

What is claimed is:

1. A hood for endoscope which is attached to an endoscope including an insertion portion inserted in a longitudinal axis direction from a distal end side into an object, a distal end constituent member that is provided on the distal end side of the insertion portion and has an opening section, and a conduit that is provided in the insertion portion and also communicatively connects an inside and an outside of the object via the opening section, the hood for endoscope comprising:
a frame mounted to an outer circumference of the distal end constituent member;
a cutout opening that is provided in a position of the frame where the cutout opening protrudes to a front side in the longitudinal axis direction from the distal end constituent member when the frame is attached to the distal end constituent member, communicatively connects an inner circumferential surface and an outer circumferential surface of the frame, and provided to be slanted to be oriented in the longitudinal axis direction; and
a straightening member with which a fluid ejected from the opening section towards the front side in the longitudinal axis direction collides and which guides the fluid from a flowing orientation of the fluid in the longitudinal axis direction to the flowing orientation of the fluid in a direction intersecting with the longitudinal axis direction in such a manner that the fluid is caused to pass through the cutout opening from an inside of the frame and flow towards an outside of the frame.

2. The hood for endoscope according to claim 1, wherein the straightening member guides the fluid ejected from the opening section in a direction different from a position of the distal end constituent member where an observation member that is provided in the distal end constituent member and also configured to observe the object is provided.

3. The hood for endoscope according to claim 1, wherein
the straightening member includes
a first straightening member with which the fluid ejected from the opening section towards the front side in the longitudinal axis direction collides and which changes the flowing orientation of the fluid from the longitudinal axis direction to the direction intersecting with the longitudinal axis direction, and
a second straightening member that guides the fluid the orientation of which is changed by the first straightening member to be caused to pass through the cutout opening from the inside of the frame and flow towards the outside of the frame.

4. The hood for endoscope according to claim 3, wherein
an air feeding hole that guides the fluid the orientation of which is changed by the first straightening member within the frame is formed in the second straightening member, and
the cutout opening is located on an extension line of the air feeding hole, and a space is provided between the cutout opening and the air feeding hole.

5. The hood for endoscope according to claim 1, wherein
the cutout opening in plurality is provided in the frame, and
the straightening member simultaneously guides the fluid towards the cutout opening in plurality.

6. The hood for endoscope according to claim 5, wherein two pieces of the cutout opening are provided in the frame to be adjacent to both arms in a circumferential direction of the frame in a section where the straightening member is provided.

7. The hood for endoscope according to claim 1, comprising:
a positioning portion that regulates an attachment position of the frame in a circumferential direction of the frame with respect to the outer circumference of the distal end constituent member.

8. The hood for endoscope according to claim 7, wherein the frame is positioned and attached to the distal end constituent member in such a manner that the cutout opening faces in any one of right and left directions in an orientation in the circumferential direction in which a direction reference is set in up and down directions of an image obtained by an observation member provided in the distal end constituent member and configured to observe the object.

9. The hood for endoscope according to claim 1, wherein at least one of the first straightening member and the second straightening member is integrally formed with the frame.

10. The hood for endoscope according to claim 9, wherein the first straightening member and the second straightening member are integrally formed with each other.

11. The hood for endoscope according to claim 1, wherein the cutout opening is provided to be slanted to cause a distal end surface side of the distal end constituent member to be oriented in the longitudinal axis direction.

12. A hood for endoscope which is attached to an endoscope including an insertion portion inserted in a longitudinal axis direction from a distal end side into an object, a distal end constituent member that is provided on the distal end side of the insertion portion and has an opening section, and a conduit that is provided in the insertion portion and also communicatively connects an inside and an outside of the object via the opening section, the hood for endoscope comprising:
a frame mounted to an outer circumference of the distal end constituent member;
a cutout opening that is provided in a position of the frame where the cutout opening protrudes to a front side in the longitudinal axis direction from the distal end constituent member when the frame is attached to the distal end constituent member, and communicatively connects an inner circumferential surface and an outer circumferential surface of the frame; and
a straightening member with which a fluid ejected from the opening section towards the front side in the longitudinal axis direction collides and which guides the fluid from a flowing orientation of the fluid in the longitudinal axis direction to the flowing orientation of the fluid in a direction intersecting with the longitudinal axis direction in such a manner that the fluid is caused to pass through the cutout opening from an inside of the frame and flow towards an outside of the frame, the straightening member being arranged with a space between the straightening member and the cutout opening.

13. An endoscope system comprising:
an endoscope including
an insertion portion inserted in a longitudinal axis direction from a distal end side into an object,
a distal end constituent member that is provided on the distal end side of the insertion portion and has an opening section, and
a conduit that is provided in the insertion portion and also communicatively connects an inside and an outside of the object via the opening section; and
a hood for endoscope including
a frame mounted to an outer circumference of the distal end constituent member,
a cutout opening that is provided in a position of the frame where the cutout opening protrudes to a front side in the longitudinal axis direction from the distal end constituent member when the frame is attached to the distal end constituent member, communicatively connects an inner circumferential surface and an outer circumferential surface of the frame, and provided to be slanted to be oriented in the longitudinal axis direction, and
a straightening member with which a fluid ejected from the opening section towards the front side in the longitudinal axis direction collides and which guides the fluid from a flowing orientation of the fluid in the longitudinal axis direction to the flowing orientation of the fluid in a direction intersecting with the longitudinal axis direction in such a manner that the fluid is caused to pass through the cutout opening from an inside of the frame and flow towards an outside of the frame,
the hood for endoscope being attached to the endoscope.

14. The endoscope system according to claim 13, wherein
the straightening member of the hood for endoscope includes
a first straightening member with which the fluid ejected from the opening section towards the front side in the longitudinal axis direction collides and which changes the flowing orientation of the fluid from the longitudinal axis direction to the direction intersecting with the longitudinal axis direction, and
a second straightening member that guides the fluid the orientation of which is changed by the first straightening member to be caused to pass through the cutout opening from the inside of the frame and flow towards the outside of the frame.
